(19) United States
(12) Patent Application Publication
(10) Pub. No.: US 2008/0305016 A1
(43) Pub. Date: Dec. 11, 2008

(54) Hanging box with built-in air freshener

(76) Inventor: David Fernandez Torres, Huercal De Almeria (ES)

Correspondence Address: JACOBSON HOLMAN PLLC, 400 SEVENTH STREET N.W., SUITE 600, WASHINGTON, DC, 20004, US

(21) Appl. No.: 11/785,052

(22) Filed: Apr. 13, 2007

(30) Foreign Application Priority Data
Aug. 9, 2006 (ES) U 200601878

(51) Int. Cl. A61L 9/12

(52) U.S. Cl. 422/123

(57) ABSTRACT

We describe a small hanging case with a built-in air freshener, of the type that are used suspended, which comprises two halves with a sensibly rectangular configuration with rounded edges, susceptible to being coupled to each other under pressure by means of tonguing and grooving, for which purpose the male half incorporates a perimeter lip in the coupling area in the form of an O-ring, and the opposite half includes two skirts that press on this lip, providing an airtight joint between both halves. The interior of the case contains a cellulose material plate, impregnated with the aroma and possibly of a different colour associated with each fragrance, with undulated edges and a central slot in a longitudinal direction, for purposes of increasing the evaporation surface area. Each of the halves includes ventilation grilles, with those of one half in the upper position and those of the opposite half in the lower position, thereby facilitating the ascending circulation of the air.

### OBJECT OF THE INVENTION

The present invention refers to a new type of air freshener consisting of a box provided with means for suspending it in the hanging position, including a system of air flow from bottom to top, between the rear cover and the front portion respectively, passing by way of a cellulose plate housed inside the hanging box, duly impregnated with fragrance, so that the air issues perfumed from the upper part of the front piece.

### BACKGROUND OF THE INVENTION

The different types of air fresheners currently known comprise those disposed in a plastic bag, which is cut open for their removal and use, while there are other large-sized table-top types, or those provided with several layers of cellulose or thin absorbing material, causing a quick evaporation of the perfume with a very limited useful life, diffusing the perfume incorrectly, whereas the hanging box with a built-in air freshener to which we have been referring, in view of the situation of the plate impregnated with perfume inside the actual box and the current of air which is originated from bottom to top and from the posterior to the anterior plane, permits the perfect discharge of the perfume with proper freshening of the environment.

### DESCRIPTION OF THE INVENTION

The hanging box with built-in air freshener that is the object of the present invention is made up of two sensibly rectangular halves with rounded edges, forming two male and female pieces, and a cellulose plate approximately 3.8 millimetres thick, the plate of which is trimmed in a very characteristic way, occupying the inner space of the hanging box. Its whole perimeter presents undulations disposed in order that the air drawn in from the lower part ascends and issues from the top part, producing the evaporation of the perfume. These undulations make the surface area of the cellulose plate larger in this evaporation area, while the actual cellulose plate incorporates an ascending rectangular groove in the middle in order that there may be evaporation in this central area too.

The cellulose absorbent material plate may be printed in a colour matching the perfume with which it is impregnated, so that it would be yellow-coloured for lemon, red for strawberry, green for pine, etc. Its perfuming is done before being housed in the hanging box.

Both halves of the hanging box, the male and female, are joined together by press fit, after first inserting the perfumed cellulose plate inside, while it may be observed that the male half of the box has a shoulder in the form of an O-ring all round the rim of the skirt of the piece, which is inserted between the two skirts of the female piece, exerting the due pressure between both pieces to achieve a perfectly airtight seal.

The male and female parts of the box will be fitted so that there will be ventilation grilles in the upper part on the front side and other ventilation grilles in the lower portion of the rear piece, whereby with this grille arrangement the air will circulate in an upward direction, entering via the lower grilles, passing through the perfumed cellulose plate, both via the central area and via the peripheral lateral areas, and finally issuing from the upper ventilation grilles, carrying the perfume with it. In view of its cleanliness and originality, this highly effective ascending and transverse air flow system in a small rectangular box, using only a cellulose plate in its interior, certainly represents a significant invention in the field of air fresheners.

### DESCRIPTION OF THE FIGURES

For a clearer understanding of the general features described above, drawings are attached that show us, represented graphically and diagrammatically, a case of a practical preferred embodiment of the hanging box with a built-in air freshener that is the object of the invention, while pointing out that, in view of the eminently informative nature of the drawings in question, the figures designed in them should be examined with an extremely comprehensive attitude and on a no way restrictive basis. In these figures there is represented the following:

FIG. 1 shows an inner front view of the hanging box corresponding to the female piece of the front portion of the hanging air freshener box, while it may be observed that a peripheral internal groove is disposed for the press-fitting of the rear male piece. The upper portion is provided with perfumed air outlet grilles as well as an upper projecting hanger in the middle for attaching the air freshener to the rear view mirror in a car, to a rod behind the wardrobe door in the home, or in any other suitable place.

FIG. 2 shows an outer front view of the female piece of the hanging box in FIG. 1, with the ventilation grilles and the upper hanger.

FIG. 3 shows an A-B sectional view of FIG. 1, with the internal coupling groove of the male rear portion.

FIG. 4 shows a sectional and an amplified close view C of FIG. 3, where we may see the internal peripheral rib and the groove where the adjustable rear piece is attached and press-fitted to form the hanging box.

FIG. 5 shows a front view of the cellulose plate with an approximate thickness of 3.8 millimetres, presenting undulations all round its outer edge and a central vertical rectangular groove, so that evaporation areas are obtained in the whole surface area of the cellulose plate.

FIG. 6 shows an inner front view of the male piece of the hanging box, where we may see the protruding rib all round its outer edge in the form of an O-ring, with an outer edge roll in the end of the projecting rib in the form of an O-ring for press-fitting to the female portion of the hanging box, while incorporating air inlet grilles disposed in the lower part of the male piece.

FIG. 7 shows an outer front view of the male piece of the hanging box according to FIG. 6, with the lower air inlet grilles.

FIG. 8 shows a longitudinal profile view of FIG. 6 with the projecting peripheral rib provided at the end of the shoulder in the form of an O-ring for attaching and fixing the female front piece.

FIG. 9 shows a D-E sectional view of FIG. 6, with the peripheral box sealing shoulder.

FIG. 10 shows a section close view F of FIG. 9, with the arrangement of the outer projecting edge roll at the end of the peripheral rib to form the box airtight sealing O-ring.

FIG. 11 shows a front view of the closed hanging box with the perfumed air freshener, provided with plastic material labels covering the ventilation grilles, with a shoulder for its detachment, besides another label with the type of perfume, brand and other details needed.

FIG. 12 shows a longitudinal profile view of hanging box represented in FIG. 11 hermetically sealed and provided with plastic adhesive labels on both sides.

FIG. 13 shows a rear view of the closed hanging box provided with the perfumed air freshener, bearing a self-adhesive plastic label covering the lower air inlet grilles, whose label, by means of its projection, is slightly detached leaving the part of the lower grilles free, besides including another label with marks and instructions for its handling.

FIG. 14 shows a G-H longitudinal section view of FIG. 11, where the sealing system between the two pieces of the hanging box may be observed with the upper hanger and the interior arrangement where the absorbent cellulose plate is housed, along with the lower grilles of one of the pieces of the hanging box for the intake of air and the upper grilles of the other piece of the hanging box to permit the outlet of perfumed air, which passes through the inner perfumed plate.

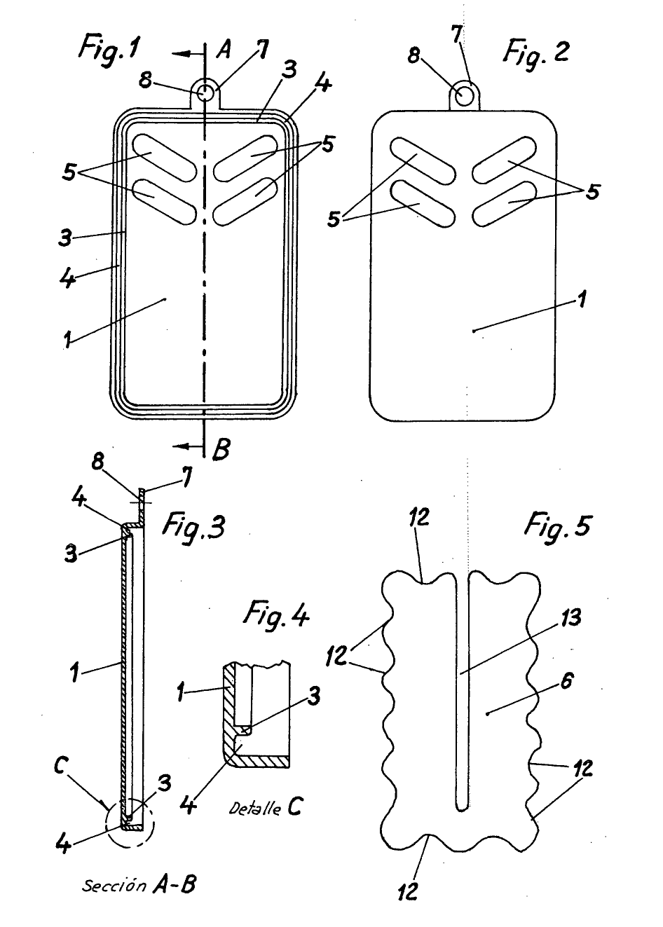

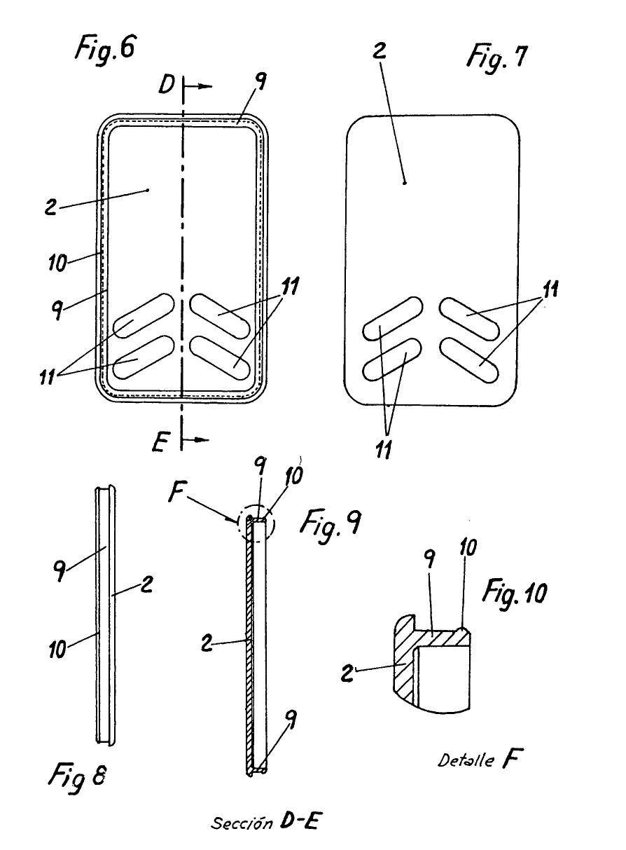

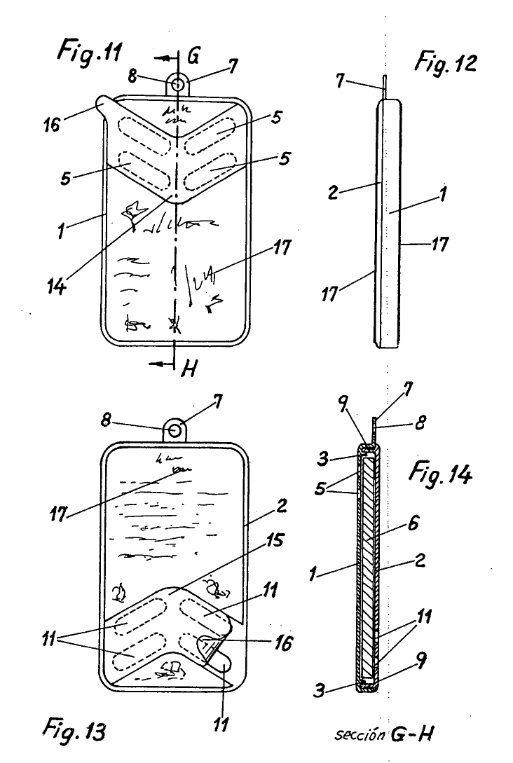

### PREFERRED EMBODIMENT OF THE INVENTION

In reference always to the adjoining drawings, it should be pointed out that in the different figures designed in them numerical elevational values are included relating to the descriptions of their features and operation given below, thereby facilitating their immediate location, (1) being the front piece of the hanging box, which is completed with the rear piece (2), both joined tightly by press-fit. On its inner plane the front piece (1) presents the lower peripheral shoulder (3) which rises from the back of the piece, forming the groove (4) all round its inner profile for fixing the rear piece (2), while the front piece (1) also incorporates in its top portion one or more through holes forming a grille (5) to permit the issue of duly perfumed air from the absorbent cellulose plate (6) housed in the hanging box. The upper part of the piece (1) is provided, exteriorly and in a central position, with a semi-circular shoulder (7) with a through hole (8), into which a multi-adaptable hanger is inserted to keep the hanging box suspended at any point at the user's discretion.

On its inner plane the rear piece (2), which forms the hanging box with piece (1), is provided with a projecting partition (9) which runs internally all the way round close to the edge of the piece (2). At the end and all round its outer edge said partition (9) incorporates a projecting lip in the form of an O-ring (10), disposed so that when the partition wall (9) of piece (2) is engaged in the groove (4) of the piece (1), a tight pressure seal is obtained, preventing the perfume with which the absorbent cellulose plate (6) is impregnated from evaporating at the sealing joint obtained between the two pieces forming the hanging box.

Like the front piece (1), the rear piece (2) will be provided with a grille (11), but in this case the grille will be located in the lower part so that the exterior air that enters through the lower grille (11) in the rear cover (2), after passing through the absorbent cellulose plate (6), impregnated with perfume may issue duly perfumed via the grille (5) in the upper part of the front piece (1) to freshen the environment.

The absorbent cellulose plate (6) housed in the hanging box between pieces (1) and (2), will have an approximate thickness of 3.8 millimetres occupying the space existing in the hanging box, and all round its outside edge it presents undulations (12), so that the air passes over them to bring about the evaporation of the perfume, as these undulations (12) make the surface area of the plate in this evaporation area larger, and for the same purpose too there is a vertical groove (13) provided in the middle of the plate (6) so that evaporation may take place in this central area as well.

In the front and rear faces of the air freshener and covering each of the grilles (5) and (11), respectively, self-adhesive plastic labels (14) and (15) are provided that keep the grilles closed, both having a tab (16) for their detachment, so that said labels (14) and (15) keep the grilles closed, and the system therefore inactive, until the time of its use, when either only one or both of the labels may be removed at the same time for utilisation of either partial or the full perfuming effect.

Besides the above-mentioned labels, each face of the hanging box will bear another label (17), in one case stating the type of perfume with the appropriate designs and, on the other side, the label (17) will inform of the instructions for use, precautions, brand, best used by date, as well as other unspecified directions.

Considering each and every one of the parts making up the hanging box with a built-in air freshener that is the object of the invention more than adequately described, it only remains for us to point out that the possibility that its different parts may be manufactured in a variety of materials, sizes, shapes and colours, while those variations of a constructional type may be introduced into its execution that practice so advocates, providing that they are not capable of altering the essential points of what is the object of the present Utility Model registration.

1. Hanging box with built-in air freshener, characterised in that it comprises two interconnectable press-fit pieces, a female front which incorporates on its inner plane a low peripheral shoulder that rises from the back of the front piece, forming a similarly peripheral groove between the small inner shoulder and the partition in the profile of the actual piece, presenting on the front plane and in its upper half one or more through holes making up a grille for the issue of the now perfumed air including, likewise in its upper and exteriorly projecting part, a small strip pierced in the centre of its width to enable the air freshener to be suspended, while the box is completed with a rear male piece which incorporates on its inner plane a projecting partition wall that runs internally all round the perimeter of the piece, close to its edge, said partition incorporating at the edge of its end and all along its outer perimeter an external protruding lip in the form of an O-ring, which fits into the groove in the female front piece when the box is closed, so that a fully airtight and watertight seal is obtained, while the rear male piece presents on its front plane and in its lower half one or more through-holes forming a grille for the intake of ambient air.

2. Hanging box with built-in air freshener according to the previous claim, characterised in that the absorbent cellulose plate housed in the box, with an approximate thickness of 3.8 millimetres, presents trimmed undulations all round its outside edge, permitting the passage of air to facilitate the evaporation of the perfume with which the cellulose plate is impregnated, while the actual plate is also provided at its centre with a vertical groove, with the result in both cases that the possibility of evaporation of the perfume in these areas may be greater, as the air flows over the undulations and along the central groove.
